(19) United States
(12) Patent Application Publication
(10) Pub. No.: US 2014/0044814 A1
(43) Pub. Date: Feb. 13, 2014

(54) Composition and Methods for Permanent Hair Removal

(71) Applicant: Rachel Brown, Eden, UT (US)

(72) Inventor: Rachel Brown, Eden, UT (US)

(21) Appl. No.: 13/938,886

(22) Filed: Jul. 10, 2013

Related U.S. Application Data
(60) Provisional application No. 61/670,068, filed on Jul. 10, 2012.
(60) Provisional application No. 61/732,733, filed on Dec. 3, 2012.

(51) Int. Cl. A61K 8/97; A61K 8/26; A61Q 7/02; A61K 8/98

(52) U.S. Cl. CPC A61K 8/97; A61K 8/986; A61K 8/26; A61Q 7/02
USPC 424/756

(57) ABSTRACT

Compositions and methods to prevent future hair growth are shown. After a skin treatment area is subjected to standard stripping procedures (such as sugaring or waxing), turmeric or a composition containing turmeric is applied to the skin treatment area. The composition is partially deposited into the generally empty hair follicles and cuts off blood supply necessary for new hair growth. After a period of time, the composition may be wiped off the skin's surface (remaining in the follicle) and oil may be applied to the skin to aid healing. Multiple treatments consisting of hair stripping and applying the turmeric composition may result in hair follicles that breakdown and are unable to grow hair again.

### BACKGROUND OF THE INVENTION

1. The Field of the Invention

The present invention relates to hair removal. More specifically, the present invention relates to a method for permanently removing unwanted hair or permanently reducing the amount of unwanted hair.

2. State of the Art

According to the prior art, it may be difficult to permanently remove unwanted hair. Procedures such as plucking, shaving, waxing, sugaring, electrolysis, and the like have been used to temporarily remove unwanted hair. However, most of these procedures must be done on a continual basis to keep unwanted hair away. Even more permanent solutions, such as laser treatment, take several sessions in order to be successful, and still cannot guarantee that unwanted hairs will not grow back.

Additionally, laser treatments cannot be used on all types of skin types because the laser targets pigment, so the treatment may be limited by hair or skin color.

In some Asian countries, traditional remedy which are been used for removing fine hairs, such as women's facial hairs (superfluous hair) and the fine hair on many newborns (lanugo hair) is turmeric. The turmeric may be mixed with a carrier to form a paste like substance and applied to the skin Once dried, the paste material may be scrubbed and the hairs are removed with the paste.

While each of the above-referenced methods helps remove some hair, there is a need for an improved hair removal technique. The method should preferably be able to be used on all skin types, and target the actual hair follicle in order to prevent future hair growth. It is also desired for the method to be relatively low in cost and simple to practice.

### SUMMARY OF THE INVENTION

It is an object of the present invention to provide an improved method for more permanent removal of hair.

In accordance with the principles of the invention, a method is provided that targets the actual hair follicle, and thus may be used on persons of all skin and hair types.

According to one aspect of the invention, hair is stripped from the treatment area, and then turmeric or a turmeric containing compound is applied to skin The turmeric containing compound may be selected from any of a variety of carrying agents including, but not limited to, milks, ointments, gels, milk and other compositions. In accordance with another aspect of the invention, the turmeric or turmeric containing compound is allowed to dry and then is removed from the top surface of the skin.

In accordance with another aspect of the invention, a southing/sealing agent is applied to the skin to help retain the turmeric or turmeric containing compound in the hair follicles.

In accordance with one aspect of the invention, the turmeric containing composition is a clay-turmeric composition which is applied to the skin and allowed to dry.

In accordance with another aspect of the invention, a composition containing turmeric and at least one of milk, soy milk and coconut milk/coconut oil is applied to skin and allowed to dry.

In accordance with another aspect of the invention, a warm moist material is applied to the skin and the composition containing turmeric is wiped off the upper surface of the skin.

In accordance with another aspect of the invention, an oil, such as olive oil, frankincense, lavender oil or tee tree oil, or mixture thereof is used as the southing/sealing agent to help trap the turmeric containing composition in the hair follicles.

According to another aspect of the invention, the steps of stripping the hair, applying a turmeric composition, wiping the area and applying oil, are repeated. The steps are repeated after hair growth is shown on the area.

These and other aspects of the present invention are realized in methods for permanent hair removal or reduction and compositions for permanent hair removal or reduction as shown and described in the following figures and related description.

### BRIEF DESCRIPTION OF THE DRAWINGS

Various embodiments of the present invention are shown and described in reference to the numbered drawings wherein:

FIG. 1 shows a cross-sectional view of a hair follicle and the surrounding tissue;

FIG. 2 shows a cross-sectional view of the hair follicle according to FIG. 1, with the hair being removed and the composition according to the present method being applied;

FIG. 3 shows a cross-sectional view of the hair follicle of FIG. 2 after the composition has been removed from the surface of the skin;

FIG. 4 shows a cross-sectional view of a hair follicle and the surrounding tissue after one or more treatments according to the present method; and

FIG. 5 shows the steps of a method for permanently removing unwanted hair.

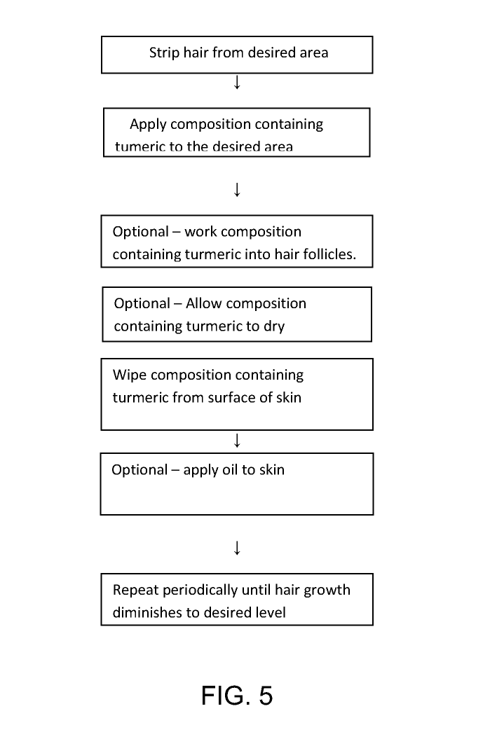

It will be appreciated that the drawings are illustrative and not limiting of the scope of the invention which is defined by the appended claims. The embodiments shown accomplish various aspects and objects of the invention. It is appreciated that it is not possible to clearly show each element and aspect of the invention in a single figure, and as such, multiple figures are presented to separately illustrate the various details of the invention in greater clarity. Similarly, not every embodiment need accomplish all advantages of the present invention.

### DETAILED DESCRIPTION

The invention and accompanying drawings will now be discussed in reference to the numerals provided therein so as to enable one skilled in the art to practice the present invention. The drawings and descriptions are exemplary of various aspects of the invention and are not intended to narrow the scope of the appended claims.

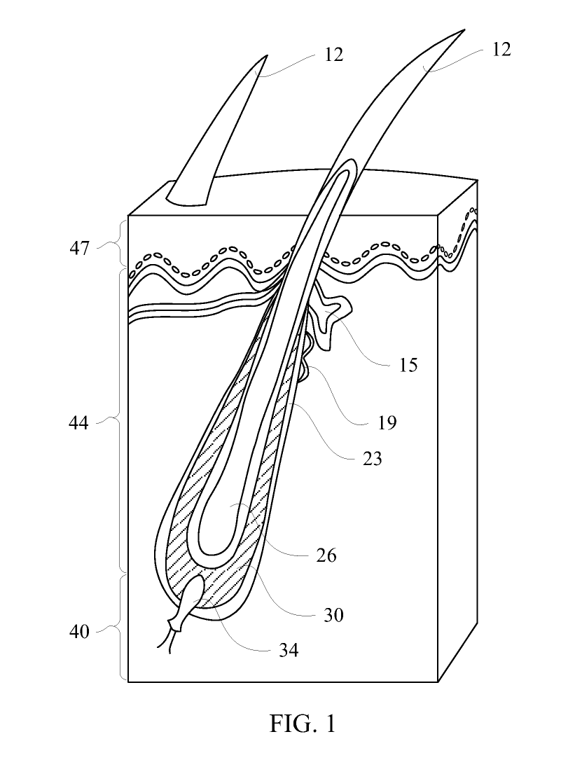

Turning now to FIG. 1, a perspective view of a hair follicle and the surrounding tissue, such as the sebaceous gland 15, bulge region 19, dermis layer 44, and subcutaneous fat layer 40. Normally, a hair follicle 26 is the organ that produces hair. Hair growth begins at the dermal papilla 34 at the base of the hair follicle 26. The visible hair 12 is seen above the epidermis layer 47 of the skin. According to one aspect of the current method, the hair in the treatment area is first removed by conventional stripping techniques, for example, sugaring, waxing, electrolysis, or laser. These techniques remove the hair from the root, leaving the follicle 26 substantially empty. Based on currently available information, sugaring and waxing appear to be preferred methods for removal of the hair from the hair follicle.

While the follicle is in this empty, generally open state, turmeric is applied to the area for which a reduction or permanent elimination of hair growth is desired. The turmeric is typically applied in a turmeric containing composition. The turmeric containing composition may include a clay-turmeric compound, a milk-turmeric compound, an ointment-turmeric compound, a gel-turmeric compound, a milk-turmeric compound, or numerous other vehicles for carrying the turmeric.

### Example 1

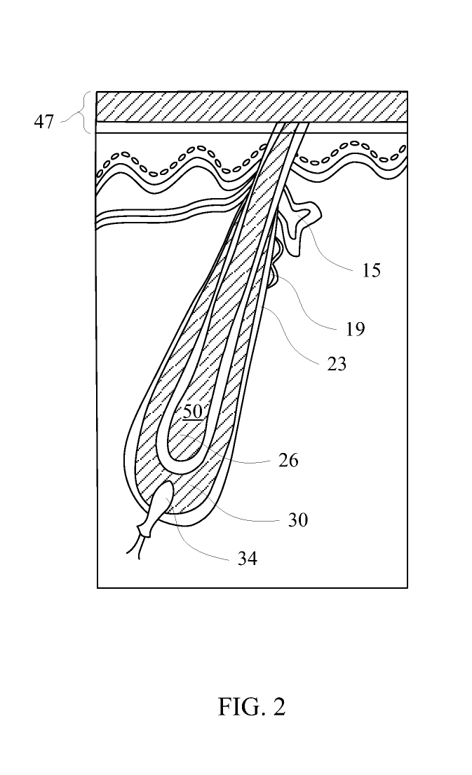

A clay-turmeric composition is spread onto the skin after the unwanted hair is removed. FIG. 2 shows the clay-turmeric composition 50 in hatching, with the composition filling the generally open follicle and being spread on the surface of the skin 47. While the clay may be spread onto the treatment area according to various methods, it may be advantageous to spread the composition in the opposite direction of the hair growth, to further ensure penetration of the composition into the empty follicle. The composition may be applied by any technique known in the art, for example, by massage or a facial masque brush. It is desirable for the composition to penetrate deeply into the follicle, so a technique such as massage or brushing in the generally opposite direction of the hair growth may be desirable.

The clay-turmeric composition includes an amount of turmeric and an amount of clay. The clay may be selected from any number of clays known in the art suitable for use on skin The mixture of clay to turmeric is variable, with more turmeric making the composition stronger, or more likely to prevent future hair growth. One example of a mixture according to the present invention is one-quarter cup of powdered Bentonite clay and one teaspoon of turmeric. This is a ratio of approximately 12 parts clay to 1 part turmeric. Other ratios such as 3 teaspoons turmeric per ¼^{th} cup clay to 1 teaspoon turmeric to one cup of clay may also be used. Water is mixed into the clay and turmeric to achieve a desirable consistency which will allow the composition to be applied to the skin Smaller amounts of turmeric may be used, especially if the treatment area has lighter, blonder hair which tends to be less persistent in growth compared to darker hair. The amount of clay to turmeric may be varied, with more turmeric making the composition more potent for denser or darker hair. However, even small amounts of turmeric, such as 1 part turmeric to 50 parts clay, may be effective depending on the type of hair. At present, it is believed that a range of about 1:12 to 1:1 turmeric to clay is preferred.

Additionally, any type of suitable clay known in the art may be used. By way of example, the clay may be chosen from any of the following types of clay: Bentonite clay, Indian healing clay, Dead Sea Mud Mineral masque, green clay, Dead Sea clay, French green clay, Sedona clay, French red clay, Montmorillonite clay, Kaolin clay, white clay, Zeolite clay, Epoch glacial marine mud, French white clay, calcium bentonite clay, China clay, French yellow clay, Moroccan red clay, rose clay, sea clay, sea weed powder, Rhassoul clay, pink Australian clay, sodium bentonite clay. Other types of clay known in the art may also be used.

It is believed that the clay-turmeric mixture works by generally penetrating the empty hair follicle and inhibiting blood flow necessary for future hair growth. As the clay-turmeric mixture inhibits blood supply to the follicle, it also may cut off the follicle from the cells that produce new hair. Over time and applications, the clay-turmeric mixture is also believed to cause the breakdown of the outer sheath of the hair follicle.

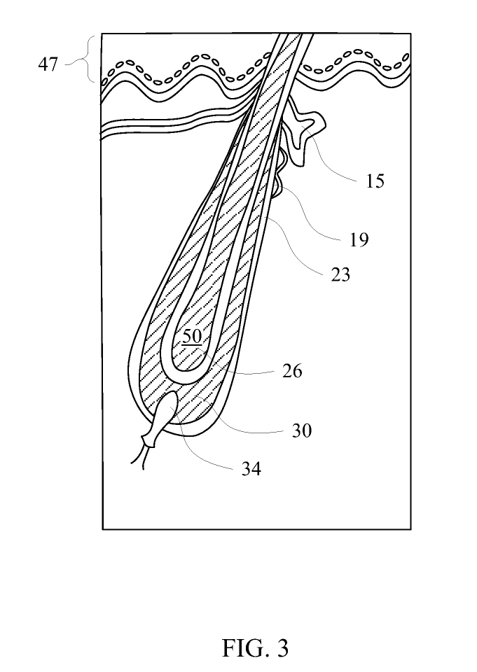

After stripping the hair on the treatment area and applying the composition of clay and turmeric, the composition is left on the skin for a period of time. For example, the composition may be left on the skin for 15-20 minutes, or until the clay has dried or substantially dried on the skin. The composition is then removed from the surface of the skin. This may be done, for example, by applying a warm, moist towel to the skin and then rubbing or wiping the clay-turmeric composition from the skin FIG. 3 shows a cross-sectional view of the hair follicle of FIG. 2, with the clay-turmeric composition 50 removed from the epidermis layer 47, but remaining within the hair follicle 26. The excess clay-turmeric composition 50 is removed from the surface of the skin, while at least some of the composition remains in the hair follicle 26.

After the composition is removed from the surface of the skin, a layer of oil, such as olive oil, frankincense, lavender oil or tee tree oil, or mixture thereof or and oil and frankincense mixture may be applied to the surface of the skin. This step may help the clay-turmeric composition left in the follicles of the treatment area to remain within the follicles longer, thus cutting off blood flow longer to prevent future hair growth. This step of applying oil may also help heal the skin which may have been aggravated by the hair stripping. Furthermore, the oil may prevent bacteria from entering the follicles and causing an infection.

One treatment may consist of the steps of stripping hair, applying a clay-turmeric composition, waiting a period of time, wiping the skin dry, and applying oil. In subsequent treatments, these steps are repeated. The time between treatments depends on how quickly the hair on the treated area grows back. For many standard stripping procedures, such as waxing, there must be sufficient growth to allow the stripping procedure to work properly. After about four treatments, permanent hair removal effects are seen (no hair will grow back on some portions of the treated area). After about six to ten treatments, total permanent hair removal may be achieved. For particularly stubborn hair, more treatments may be required, such as fifteen treatments.

It will be appreciated that controlling the number of treatments may be used to obtain a desired effect. For example, some women have excess hair on the forearm. While it may be desirable to reduce the amount of hair, some women may not want to eliminate all hair from the arms, as it may appear somewhat unnatural. Thus, the process may be repeated only 4 or 5 times to reduce the number of hairs and the thickness of the hair which do regrow without eliminating all air from the arm.

### Example 2

A milk-turmeric composition is spread onto the skin after the unwanted hair is removed. FIG. 2 may be viewed as showing the milk-turmeric composition 50 in hatching, with the composition filling the generally open follicle and being spread on the surface of the skin 47. While the milk may be spread onto the treatment area according to various methods, it may be advantageous to spread the composition in the opposite direction of the hair growth, to further ensure penetration of the composition into the empty follicle. The composition may be applied by any technique known in the art, for example, by massage or a facial masque brush. It is desirable for the composition to penetrate deeply into the follicle, so a technique such as massage or brushing in the generally opposite direction of the hair growth may be desirable.

The milk-turmeric composition includes an amount of turmeric and an amount of milk. For example 1-4 parts turmeric may be mixed with 1-4 parts milk to give a ratio of between about 1:4 to 4:1. The mixture of milk to turmeric is variable, with more turmeric making the composition stronger, or more likely to prevent future hair growth. One example of a mixture according to the present invention is 2 tsp. of milk per 1 tsp. of turmeric. This ratio of approximately 2 parts milk to 1 part turmeric is advantageous due to the consistency of the mixture. Other ratios such as 3 teaspoons turmeric per ¼^{th} cup milk to 1 teaspoon turmeric to one cup of milk may also be used. If the milk is too thick, water or another thinning agent may be mixed into the milk and turmeric to achieve a desirable consistency which will allow the composition to be applied to the skin Smaller amounts of turmeric may be used, especially if the treatment area has lighter, blonder hair which tends to be less persistent in growth compared to darker hair. The amount of milk to turmeric may be varied, with more turmeric making the composition more potent for denser or darker hair. However, even small amounts of turmeric, such as 1 part turmeric to 50 parts milk. The milk-turmeric composition may be applied in a similar manner as discussed above.

### Example 3

A cream-turmeric composition may be spread onto the skin after the unwanted hair is removed. FIG. 2 may be viewed as showing the cream-turmeric composition 50 in hatching, with the composition filling the generally open follicle and being spread on the surface of the skin 47. While the cream may be spread onto the treatment area according to various methods, it may be advantageous to spread the composition in the opposite direction of the hair growth, to further ensure penetration of the composition into the empty follicle. The composition may be applied by any technique known in the art, for example, by massage or a facial masque brush. It is desirable for the composition to penetrate deeply into the follicle, so a technique such as massage or brushing in the generally opposite direction of the hair growth may be desirable.

The cream-turmeric composition includes an amount of turmeric and an amount of cream. For example 1-10 parts turmeric may be mixed with 1-10 parts cream. The mixture of cream to turmeric is variable, with more turmeric making the composition stronger, or more likely to prevent future hair growth. One example of a mixture according to the present invention is 8 parts cream to 2 parts turmeric. Another example is 9 parts cream to 1 part turmeric. Still another example is about 1 part cream to one part turmeric. In a presently preferred range, the quantity of cream is above that of the turmeric. Other ratios such as 3 teaspoons turmeric per ¼^{th} cup cream to 1 teaspoon turmeric to one cup of cream may also be used. If the cream is too thick, water or another thinning agent may be mixed into the cream and turmeric to achieve a desirable consistency which will allow the composition to be applied to the skin Smaller amounts of turmeric may be used, especially if the treatment area has lighter, blonder hair which tends to be less persistent in growth compared to darker hair. The amount of cream to turmeric may be varied, with more turmeric making the composition more potent for denser or darker hair. However, even small amounts of turmeric, such as 1 part turmeric to 50 parts cream, can be used, although they are presently believed to be less effective than at least 1:10 and more preferably between 1:4 and 1:1. The cream-turmeric composition may be applied in a similar manner as discussed above.

### Example 4

A gel-turmeric composition may be spread onto the skin after the unwanted hair is removed. FIG. 2 may be viewed as showing the cream-turmeric composition 50 in hatching, with the composition filling the generally open follicle and being spread on the surface of the skin 47. While the gel may be spread onto the treatment area according to various methods, it may be advantageous to spread the composition in the opposite direction of the hair growth, to further ensure penetration of the composition into the empty follicle. The composition may be applied by any technique known in the art, for example, by massage or a facial masque brush. It is desirable for the composition to penetrate deeply into the follicle, so a technique such as massage or brushing in the generally opposite direction of the hair growth may be desirable.

The gel-turmeric composition includes an amount of turmeric and an amount of gel. For example 1-4 parts turmeric may be mixed with a 1-50 parts gel. The mixture of gel to turmeric is variable, with more turmeric making the composition stronger, or more likely to prevent future hair growth. One example of a mixture according to the present invention is one-part turmeric to 9 parts gel. A more preferred range is about 1 part turmeric to 4 parts gel, with a more preferred range being about 1 part turmeric to about 1 part gel. If the gel is too thick, water or another thinning agent may be mixed into the milk and turmeric to achieve a desirable consistency which will allow the composition to be applied to the skin Smaller amounts of turmeric may be used, especially if the treatment area has lighter, blonder hair which tends to be less persistent in growth compared to darker hair. The amount of gel to turmeric may be varied, with more turmeric making the composition more potent for denser or darker hair. However, even small amounts of turmeric, such as 1 part turmeric to 50 parts gel may be used. The gel-turmeric composition may be applied in a similar manner as discussed above.

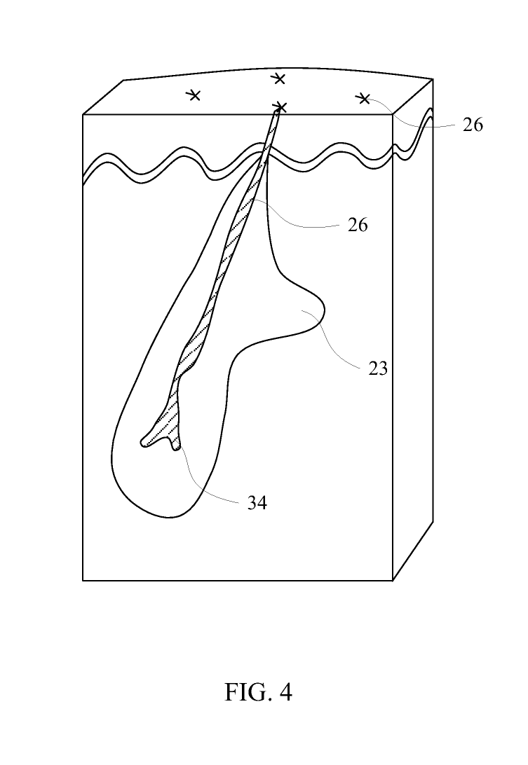

Turning now to FIG. 4, there is seen a cross-sectional view of a hair follicle that has been subjected to one or more treatments according to the present composition and method. There are no surface hairs 12, but rather empty follicles 26. The blood flow to the dermal papilla 34 has been cut-off, and the outer sheath 23 is breaking down. Under these conditions, it may be very difficult or impossible for new hair growth. Thus, follicles subjected to the compositions and methods of the present invention are generally prevented from growing new hair.

While discussed above with respect to a clay-turmeric mixture, it will be appreciated that turmeric alone or a composition of turmeric mixed with one or more of milk, soy milk, coconut milk, cream, ointment, oil or any other number of carriers can be used in accordance with the art. Example of likely combinations includes 1-4 parts turmeric and 1-50 parts milk; 1-4 parts turmeric and 1-50 parts soy milk; 1-4 parts turmeric and 1-50 parts coconut milk. A less viscous material may be beneficial as it may result in the turmeric-containing composition quickly penetrating the hair follicle, especially where the hair follicle is relatively small. However, the less viscous composition is also more likely to be drawn away from hair follicle when the composition is wiped off. Thus, skilled technicians will develop preferences for which composition to use based on the particular person being treated.

Thus, it will be appreciated that the presently described system and methods allow for the same reduction and/or cessation of hair growth and can be used safely on anyone, regardless of hair type or skin color, because it targets the actual hair follicle, rather than pigment. By regulating the number of applications, the extent of reduction in hair growth can be tailored to the individual desires of a patient. Additionally, the method does not require any expensive equipment, such as lasers, and may be done by those having relatively little training. The method is also relatively simple to use and the composition has generally available ingredients.

In addition to the above, it has been found that sea salt may also be added to the mixture. It is believed that the sea salt help to open or penetrate the cellular wall. It also appears to help to minimize folliculitis after treatment. It is believed that the sea salt should be between about 1-10 parts of the turmeric mixture, with the remaining 90-99 parts being turmeric and milk/clay/etc. A presently preferred range is about 1-2 parts sea salt to about 98-100 percent turmeric mixture.

The treatment method according to another aspect of the invention further comprises the step of wrapping the clay-turmeric composition on the skin area in a moisture impermeable barrier, such as, for example, a thin plastic film that has low permeability to water vapor and oxygen, such that the clay-tumeric composition is maintained in contact with the skin for a sufficient period of time to allow the composition to have its intended effect. Examples of suitable thin plastic films include polymers made from vinylidene chloride, polyvinylidene chloride or polyethylene, such as Saran® plastic film wrap (Dow Chemical, Michigan, USA). In some embodiments, the step of wrapping the clay-tumeric composition on the skin area in a moisture impermeable barrier is for a duration of about 1.5 hours or greater, or for a duration of about 2 hours or greater.

There is thus disclosed an improved method for achieving permanent hair reduction or permanent hair removal. It will be appreciated that numerous changes may be made to the present invention without departing from the scope of the claims. The appended claims are intended to cover such modifications.

What is claimed is:

1. A method for preventing future hair growth, the method comprising:
applying a topical application of turmeric or a turmeric containing composition to skin from which hair has been stripped before any noticeable regrowth has occurred.

2. The method according to claim 1, wherein the method comprises applying the turmeric containing composition to an area of skin having excess or unwanted hair growth and allowing the turmeric containing composition to dry.

3. The method according to claim 1, wherein the turmeric or turmeric containing composition is worked over an area of the skin so as to encourage at least some of the turmeric or turmeric containing composition to be deposited in the hair follicles below an outer surface of the skin and applying oil to the skin to help retain the turmeric or turmeric containing composition in the hair follicle.

4. The method according to claim 1, wherein step of applying a topical application of turmeric or turmeric containing composition comprises applying a mixture of turmeric and clay to the skin.

5. The method according to claim 1, wherein step of applying a topical application of turmeric or turmeric containing composition comprises applying a mixture of turmeric and at least one of milk, soy milk, coconut milk, coconut oil, gel, cream and ointment.

6. The method according to claim 1, wherein the method comprises stripping hair from the skin by using sugaring or waxing immediately before applying the turmeric or turmeric containing composition.

7. The method according to claim 1, wherein the turmeric or turmeric containing composition is allowed to dry on the skin.

8. A method for reducing regrowth of hair comprising applying a topical application of a clay-turmeric composition comprising turmeric and clay to an rear of skin having unwanted hair growth.

9. The method according to claim 8, wherein the method further comprises leaving the topical application of the clay-turmeric composition on the skin area for a period of time.

10. The method according to claim 9, wherein the period of time is approximately 10 to 15 minutes.

11. The method according to claim 9, wherein the topical application of the clay-turmeric composition comprises massaging or rubbing the clay-turmeric composition on the skin area in a direction against hair growth.

12. The method according to claim 9, wherein the method further comprises removing the clay-turmeric composition from the surface of the skin after a time, and leaving behind the clay-turmeric composition that may remain in a plurality of hair follicles.

13. The method according to claim 12, wherein the method further comprises topical application of an oil following removal of the clay-turmeric composition from the surface of the skin.

14. The method according to claim 13, wherein the oil comprises a mixture of oils.

15. The method according to claim 14, wherein the mixture of oils is comprised of two or more of the following types of oils: olive oil, lavender oil, tea tree oil, eucalyptus oil.

16. The method according to claim 13, wherein the application of oil comprises applying a mixture of oil and frankincense.

17. The method according to claim 8, wherein the clay of the clay-turmeric composition is formed from one or more of the following types of clay: Bentonite clay, Indian healing clay, Dead Sea Mud Mineral masque, green clay, Dead Sea clay, French green clay, Sedona clay, French red clay, Montmorillonite clay, Kaolin clay, white clay, Zeolite clay, Epoch glacial marine mud, French white clay, calcium bentonite clay, China clay, French yellow clay, Moroccan red clay, rose clay, sea clay, sea weed powder, Rhassoul clay, pink Australian clay, sodium bentonite clay.

18. A composition for reducing future hair growth comprising an amount of clay and an amount of turmeric.

19. The composition according to claim 18, wherein the ratio of clay to turmeric is from about 10 parts clay to 1 part turmeric, to about 20 parts clay to 1 part turmeric.

20. A treatment method for preventing future hair growth on a skin area, one treatment comprising:
subjecting the skin area to a hair-stripping technique;
applying a clay-turmeric composition to the skin area in the opposite direction of the hair growth;
leaving the clay-turmeric composition on the skin area for a period of time;
wiping the clay-turmeric composition from the a surface of the skin area; and
applying an oil to the skin treatment area.

21. The treatment method according to claim 20, wherein the treatment is repeated between four and fifteen times.

22. The treatment method according to claim 21, wherein the treatment is only repeated after the skin treatment area has shown an amount of hair growth.

23. The treatment method according to claim 20, wherein applying oil to the skin treatment area comprises applying a mixture of oil and frankincense.

24. The treatment method according to claim 20, further comprising the step of wrapping the clay-turmeric composition on the skin area in a moisture impermeable barrier.

25. The treatment method according to claim 24, wherein the step of wrapping the clay-tumeric composition on the skin area in a moisture impermeable barrier is for a duration of about 1.5 hours or greater.

26. The treatment method according to claim 24, wherein the step of wrapping the clay-tumeric composition on the skin area in a moisture impermeable barrier is for a duration of about 2 hours or greater.
